(19) United States
(12) Patent Application Publication
(10) Pub. No.: US 2009/0254125 A1
(43) Pub. Date: Oct. 8, 2009

(54) Top Loading Polyaxial Spine Screw Assembly With One Step Lockup

(76) Inventor: Daniel Predick, Chicago, IL (US)

Correspondence Address: Bruce J. Bowman;Bowman & Associates, Suite 106, 1016 3rd Ave. SW, Carmel, IN, 46032, US

(21) Appl. No.: 12/417,379

(22) Filed: Apr. 2, 2009

Related U.S. Application Data
(60) Provisional application No. 61/072,857, filed on Apr. 3, 2008.

(51) Int. Cl. A61B 17/70; A61B 17/86

(52) U.S. Cl. 606/264; 606/278; 606/302

(57) ABSTRACT

A top loading polyaxial spinal rod screw assembly is configured for simultaneous or one step lockup of angular orientation of a rod holder portion of the screw assembly relative to a bone screw and of a spinal rod within the rod holder portion of the screw assembly. The spinal rod and the angular orientation of the rod holder holding the spinal rod are thus fixed upon receipt of a lockup component of the present assembly. Components of the present spinal rod screw assembly cooperate to allow reception of a spinal rod in a rod holder that is adapted for pivotal retention on a bone screw and to lock or fix the spinal rod within the rod holder while also locking or fixing the angular orientation of the rod holder relative to the bone screw upon receipt of a lockup cap component of the screw assembly in the rod holder. In one form, the present screw assembly is defined by a three-piece polyaxial screw head assembly for a bone screw. The three-piece polyaxial screw head assembly includes a spine rod holder or head configured for pivotal connection with the bone screw, a head insert configured for reception in the spine rod holder, and a lockup (end) cap configured for reception in the spine rod holder. The lockup cap is configured to interact with the head insert to fix an angular position or orientation of the spine rod holder relative to the bone screw at the same time as fixing a spinal rod that has been received in the spine rod holder relative to the spine rod holder.

### RELATED APPLICATIONS

This patent application claims the benefit of and/or priority to U.S. Provisional Patent Application Ser. No. 61/072,857 filed Apr. 3, 2008, entitled “Top Loading Polyaxial Spine Screw Assembly With One Step Lockup” the entire contents of which is specifically incorporated herein by this reference.

### BACKGROUND OF THE INVENTION

1. Field of the Invention

The present invention relates to spine fixation components, constructs and assemblies and, more particularly, to screw assemblies for spinal rod applications.

2. Background Information

Spinal orthopedic assemblies and constructs such as spine plates, spinal bone screw assemblies for spinal rods and other devices (spinal components) have made a profound contribution to the correction of spinal deformities, accidents and other problems in the cervical as well as thoracic, lumbar and sacral spine. These and other spinal devices are typically fixed to vertebrae using vertebral bone screws. Vertebral bone screws are specially designed and manufactured bone screws that are placed into the bone of a vertebra. Vertebral bone screws placed in the vertebra offer superior strength and pull-out resistance as compared to other forms of fixation in spine surgery. The ability to achieve vertebral fixation has allowed surgeons to obtain more secure fixation of the spinal components involved, which permits more powerful correction of spine problems and reported better clinical outcomes.

In addition to other uses, bone screws provide a solid foundation for the attachment of spinal rods. Spinal rods are used for the fixation of a plurality of vertebrae for various situations. A spinal rod is held relative to the vertebrae by a spinal rod screw assembly. Various types of spinal rod screw assemblies are known such as those that allow for inter-operative adjustments in the coronal, transverse and sagittal planes. Certain spinal rod screw assemblies allow for various degrees of freedom of attachment of a spinal rod thereto from any direction, angle, and height. In all cases, however, the spinal rod screw assemblies hold a spinal rod and are fixed to a vertebra.

Spinal rods can thus be rigidly locked into a variety of positions along with other types of implant components. This allows a surgeon to tailor-make each construct for the individual case. However, locking of the various components of current spinal rod screw assemblies requires several steps and/or features or mechanisms thereof. This increases operative time by the surgeon to install the spinal rod screw assembly.

In view of the above, there is a need for a spine rod screw assembly that is easier to install. Overall, there is room for improvement of current spinal rod screw assemblies.

### SUMMARY OF THE INVENTION

The present invention is a top loading polyaxial spinal rod screw assembly that is configured for simultaneous, one step lockup of angular orientation of a rod holder of the screw assembly relative to a bone screw and a spinal rod within the rod holder of the screw assembly. In this manner, the spinal rod and the angular orientation of the rod holder holding the spinal rod is fixed upon receipt of a locking component of the present assembly in the rod holder.

Particularly, components of the present spinal rod screw assembly cooperate to allow reception of a spinal rod in the rod holder that is pivotally retained on a bone screw and to lock or fix of the spinal rod within the rod holder while also locking or fixing the angular orientation of the rod holder relative to the bone screw upon receipt of a locking cap component in the rod holder.

In one form, the present spinal rod screw assembly comprises a three piece polyaxial screw head assembly for a bone screw. The three-piece polyaxial screw head assembly includes a spine rod holder or head configured for pivotal connection with the bone screw, a head insert configured for reception in the spine rod holder, and an end cap configured for reception in the spine rod holder. The end cap is configured to interact with the head insert to fix an angular position or orientation of the spine rod holder relative to the bone screw at the same time as fixing a spinal rod that has been received in the spine rod holder relative to the spine rod holder.

In particular, the head of the bone screw is pivotally held by the rod holder while the shank of the bone screw extends from an end of the rod holder. The insert or collet is received within a bore of the rod holder, the lower part of the wall of which angles radially inwardly. The insert has a shape that cooperates with the bore wall such that downward pressure on the insert by the lockup cap provides axial and radial pressure on the bone screw head through a lower portion of the insert while providing axial and radial pressure on the spinal rod by upper portions of the insert.

In one form, the insert has threads on its external surface that match with threading on the bore wall of the rod holder. The insert is thus threadedly received by the rod holder via a top portion of the rod holder. The insert and rod holder are configured to provide fixation of bone screw orientation relative to the rod holder and of a spine rod within the rod holder upon receipt of the lockup cap by the rod holder.

The lockup cap or end is preferably, but not necessarily, installed as part of a lockup cap assembly which includes a lockup cap having a detachable driver portion thereon for installing the lockup cap into the rod holder. The detachable driver portion may comprise a detachable hex portion for receiving a hex driver. The hex portion is connected to the lockup cap such that an amount of rotational force (torque) detaches or removes the hex portion from the collar. An appropriate amount of torque is applied by the hex driver as the lockup cap bottoms out on the insert within the rod holder. The hex portion remains in the hex driver after detachment and is then retrieved therefrom after installation.

The present spine rod screw assembly is for use on the posterior side of the spine. Also, the present spine rod screw assembly is for use in all areas of the spine.

The present spinal rod screw assembly is formed of one or more biocompatible materials suitable for spine implants. Various sizes of the present spinal rod screw assembly are manufactured for various applications.

### BRIEF DESCRIPTION OF THE DRAWINGS

The above mentioned and other features, advantages and objects of this invention, and the manner of attaining them, will become apparent and the invention itself will be better understood by reference to the following description of an embodiment of the invention taken in conjunction with the accompanying drawings, wherein:

FIG. 1 is an exploded perspective view of the components of a spinal rod attachment assembly fashioned in accordance with the present principles;

FIG. 2 is a sectional view of an upper portion of the spinal rod attachment assembly of FIG. 1, assembled;

FIG. 3 is a side view of the spinal rod attachment assembly of FIG. 1 holding a spinal rod therein and having received a breakaway collar thereon for fixing a spinal rod in and the spinal rod attachment assembly;

FIG. 4 is a sectional view of the spinal rod attachment assembly of FIG. 3 taken along line 4-4 thereof;

FIG. 5 is a sectional view of the spinal rod attachment assembly of FIG. 3 akin to the line taken along 5-5 thereof but with the breakaway collar in a fully downward position; and

FIG. 6 is the sectional view of FIG. 5 with a removable portion of the breakaway

Like reference numerals indicate the same or similar parts throughout the several figures.

A description of the features, functions and/or configuration of the components depicted in the various figures will now be presented. It should be appreciated that not all of the features of the components of the figures are necessarily described. Some of these non discussed features as well as discussed features are inherent from the figures. Other non discussed features may be inherent in component geometry and/or configuration.

### DETAILED DESCRIPTION OF AN EMBODIMENT OF THE INVENTION

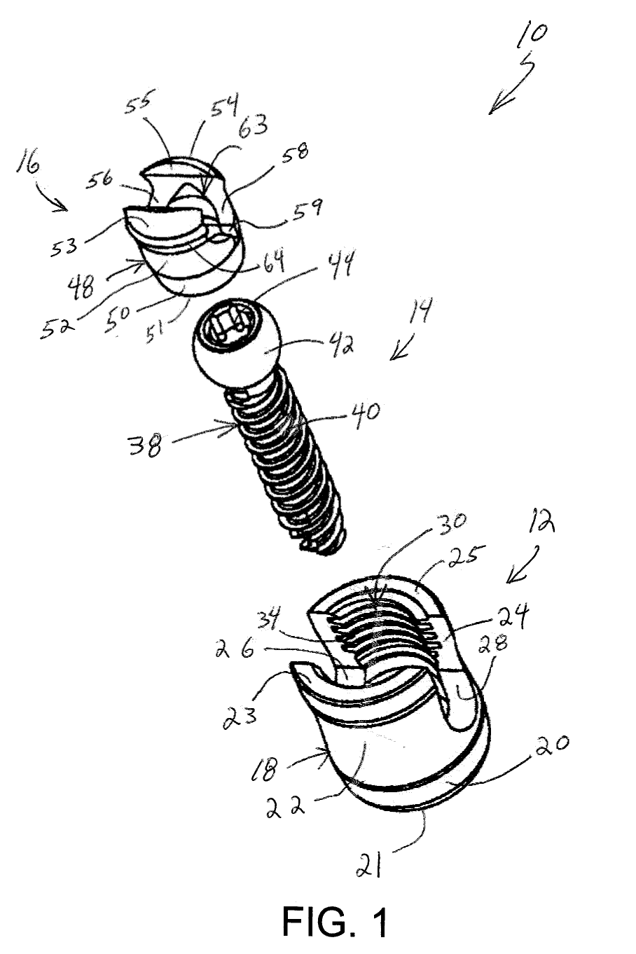

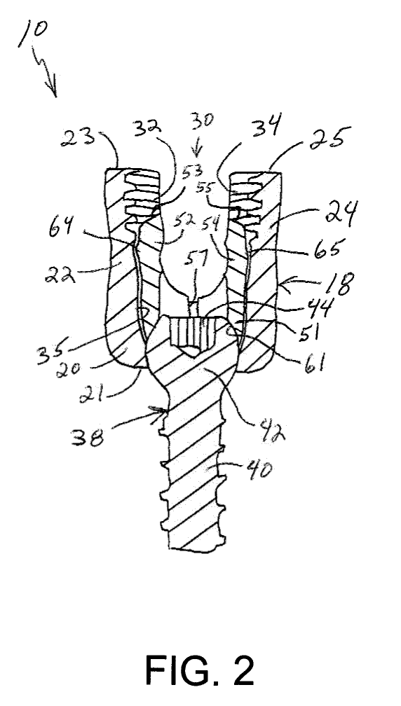

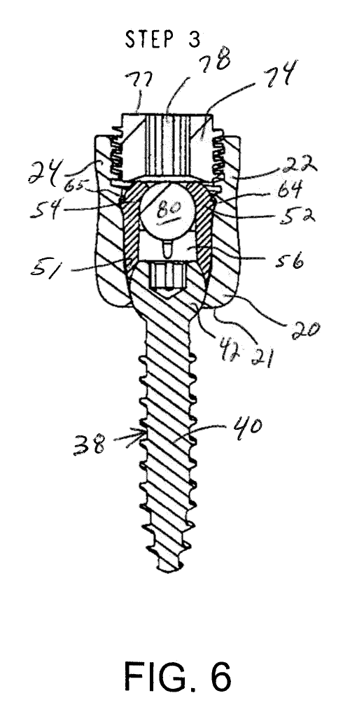

FIGS. 1 and 2 depict various views of a polyaxial spinal rod screw system or assembly 10 for holding a spinal rod relative on and to a vertebra (not shown). FIG. 1 depicts base components of the spinal rod screw assembly 10 in an exploded or pre-assembled view. FIG. 2 is a sectional view of the components of the screw assembly 10 in an assembled state. The spinal rod screw assembly 10 in one form is characterized by a rod holder, connector or head 12, an insert or collet 16, and a collar, end cap, lockup cap or the like 74. The spinal rod screw assembly in another form is characterized by a rod holder, connector or head 12, a bone screw 14, an insert 16, and a lockup cap 74 (see, e.g., FIG. 6). In another form, the spinal rod screw assembly is characterized by a rod holder, connector or head 12, an insert or collet 16, and a bone screw 14. In a further form, the spinal rod screw assembly is characterized by a rod holder, connector or head 12, an insert or collet 16, a bone screw 14, and an end cap, lockup cap or collar 74. Other permutations are contemplated.

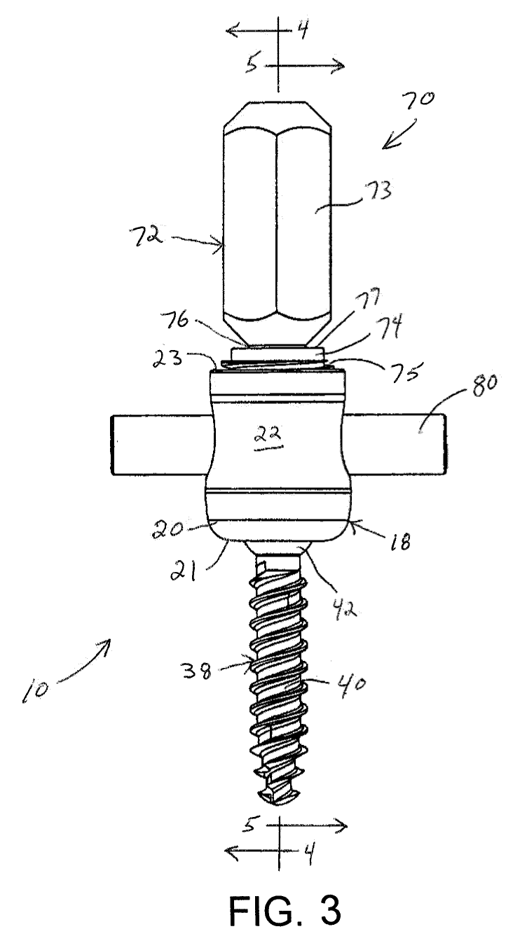

The rod holder 12 is defined by a generally tulip shaped body 18, it being understood that the body 18 may take other shapes as appropriate. The body 18 has a base 20 defining a bottom 21, a first side or sidewall 22 extending from one side of the base 20 and a second side or sidewall 24 extending from another side of the base 20. The first sidewall 22 defines a first upper end 23 while the second sidewall 24 defines a second upper end 25. A first pocket, cutout or notch 26 is provided between the first and second sides 22, 24 on a yet another side of the base 20, while a second pocket, cutout or notch 28 is provided between the first and second sides 22, 24 on still another side of the base 20. The first and second notches 26, 28 are formed to receive a spinal rod therein (see, e.g. FIGS. 3-6) and thus are generally arcuate or cup-shaped.

The body 18 includes a bore 30 defining a cavity therein that is in communication with one side of each notch 26, 28. The first sidewall 22 has screw threads 32 on an inner surface thereof, while the second sidewall 24 likewise has screw threads 34 on an inner surface thereof. The first and second screw threads 32, 24 are adapted to threadedly receive a threaded collar 74 (see, e.g., FIGS. 3-6).

As seen in FIG. 2, the inner wall 61 of axially lower portion of the bore 30 is angled or tapered inwardly and ends in an opening through which the bone screw 14 extends. The wall configuration of the bore 30 and the opening at the bottom 21 of the body 18 captures or holds the bone screw 14 for pivotal movement therein and relative to.

The bone screw 14 is characterized by a body 38 defining a threaded shaft or shank 40 with a generally rounded screw head 42 at one end of the threaded shaft 40. The threaded shaft 40 terminates at an end opposite the screw head 42 in a general point configured for reception in a vertebra. The head 42 has a socket 44 therein (see, e.g. FIG. 2) for reception of a bone screw driver (not shown). The socket 44 is preferably configured to receive a like configured bone screw driver. In one form, the socket 44 is configured as a hex and as such a hex bone screw driver is utilized to drive and reverse drive the bone screw 14.

The rod holder insert (insert) or collet 16 is defined by a generally truncated tulip shaped body 48 sized to be operatively received in the rod holder 12. The body 48 has a base or lower portion 50 having a lower end 51 with a first side or sidewall 52 axially extending from one side of the base 50 and a second side or sidewall 54 axially extending from another side of the base 50 preferably but not necessarily such that the first and second sidewalls 52, 54 are opposite one another. The first sidewall 52 has a first upper end 53 while the second sidewall 54 has a second upper end 55. A first pocket, cutout or notch 56 is provided between one side of the first and second sides 52, 54, while a second pocket, cutout or notch 58 is provided between another side of the first and second sides 52, 54. Preferably, but not necessarily, the first and second notches 56, 58 are opposite one another. The first and second notches 56, 58 are formed to receive a spinal rod therein (see, e.g. FIGS. 3-6) and thus are generally arcuate or cup-shaped.

The body 48 includes a bore 63 that defines a cavity therein which is in communication with one side of each notch 56, 58. As seen in FIG. 2, the generally annular end 51 is shaped or curved to radially encompass the head 42 of the bone screw 14. The angular wall configuration 61 of the bore 30 and the lower shape or end 51 of the insert 16 creates wedging and/or radial construction of the end 51 of the insert 16 creating axial pressure or compression against and/or onto the head 42 of the bone screw 14. When the insert 16 is axially downwardly forced or compressed (such as when installed with an end or lockup cap or collar) the body 48 thereof fixes pivoting motion of the bone screw 14 relative to the rod holder 12, thereby fixing the angular orientation of the bone screw 14 relative to the rod holder 12.

Moreover, the body 48 has a knob or ridge 64 is provided on the outside of the sidewall 52 proximate the end 53 thereof. A knob or ridge 65 is also provided on the outside of the sidewall 54 proximate the end 55 thereof. The knobs 64, 65 cooperate with the angled walls of the rod holder bore 30 promoting radial compression of the body 48 against the spinal rod 80, promoting radial compression of the rod.

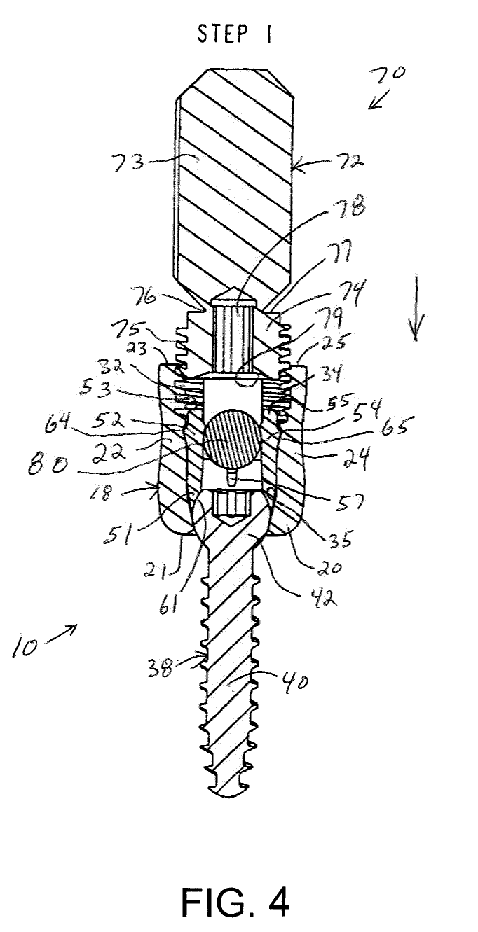

Upon insertion of the spinal rod 80 within the rod holder 12, the interference fit of the collect splays slightly, allowing the spinal rod 80 to remain captured in the collet pocket. Also, as best seen in the sequence of FIGS. 4-6, the angular wall configuration 61 of the bore 30 and the configuration of the first and second sidewalls 52, 54 of the insert 16 provide radial and axial pressure against the spinal rod 80 installed within the rod holder 12 when downwardly axially compressed by the collar 74. Resultantly, the bottom surface of the spinal rod 80 is additionally axially compresses downward onto surfaces 26 and 28 of the rod holder 12 while remaining proud of the screw head 42.

The collar 74 has a generally annular body with external threads 75 thereon that match the threads 32 and 34 of the first and second sidewalls 22, 24 of the rod holder 12. The lockup cap 74 is preferably, but not necessarily, part of a cap assembly 70 configured to allow installation of the cap 74 thereof onto the rod holder 12. The collar assembly 70 includes the cap 74 and a detachable portion 72 connected to an upper end 77 of the cap 74. The detachable portion 72 is formed as a hex head 73. A reduced diameter or scored portion 76 defines a junction or juncture 76 between the hex head 73 and the upper end 77 of the cap 74. The junction 76 allows the hex head 73 to be removed from the cap 74 upon the application of a proper amount of torque.

Referring to FIG. 3, a spine rod or portion of a spine rod 80 is shown received in a spinal rod screw assembly 10 as would be the case when being installed or implanted on a vertebra. The spinal rod 80 extends through the notches 26, 28 of the rod holder 12. The cap assembly 70 is also shown situated in and/or on the rod holder 12.

FIGS. 4-6 depict steps 1-3 in installing or using the present screw assembly 10. FIG. 4 may be considered Step 1 (and is labeled as such) of a three step process that provides single, concerted or simultaneous lockup of the spine rod 80 in and relative to the rod holder 12 and the orientation of the bone screw 14 relative to the rod holder 12. In Step 1, the spine rod 80 is received in the rod holder 12, while the bone screw 14 is axially oriented relative to the rod holder 12. The cap assembly 70 has been received in the rod holder and particularly the threaded insert 74 is threadedly received by the threads 32, 34 of the sidewalls 22, 24 of the rod holder body 18.

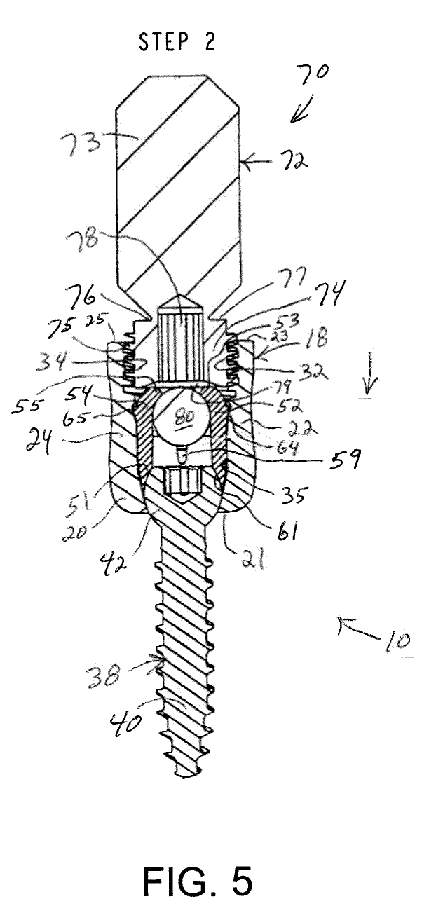

FIG. 5 may be considered Step 2 (and is labeled as such) of the present three step process. The cap assembly 70 has been rotatably driven downwardly into the rod holder 12 through application of a hex driver (not shown) such that the end 79 (see FIG. 4) of the cap 74 bottoms out or compressed against the ends 53, 55 of the sidewalls 52, 54 of the insert 16. At this point the lockup is complete as the cap, insert, and rod holder cooperate as described above to simultaneously or in one step, fix and retain the bone screw and the spinal rod.

FIG. 6 may be considered Step 3 (and is labeled as such) or the last step of the present three step process. The hex head 73 has been removed or broken away from the cap assembly leaving the cap 74 in the rod holder 12. A configured socket (e.g. a hex socket) 78 of the cap 74 is now exposed for removing the cap 74, further tightening or readjustment if necessary.

The various components of the present spinal rod screw assembly 10 are made from a bio-compatible material such as stainless steel or titanium. Other bio-compatible materials, or course, may be used.

While the invention has been illustrated and described in detail in the drawings and foregoing description, the same is to be considered as illustrative and not restrictive in character, it being understood that only a preferred embodiment has been shown and described and that all changes and modifications that come within the spirit of the invention are desired to be protected.

What is claimed is:

1. A spinal rod reception assembly for a spinal screw, the spinal rod screw assembly comprising:
a spinal rod holder configured to hold a spinal rod and having an axial bore therein defining a first opening and a second opening, the second opening configured to hold a head portion of a spinal screw and to allow polyaxial pivoting of the spinal screw, the first opening configured to receive the spinal rod;
an insert received in the axial bore of the spinal rod holder and configured to receive the spinal rod, the insert defining a first end and a second end, the second end configured to abut the head portion of the spinal screw and to fix an angular orientation of the spinal screw relative to the spinal rod holder upon application of axial pressure on the first end of the insert, and the first end configured to fix a position of the spinal rod relative to the spinal rod holder upon application of the axial pressure on the first end of the insert; and
an end cap receivable in the axial bore of the spinal rod holder and configured to apply axial pressure on the first end of the insert after placement of the spinal rod in the spinal rod holder to fix the angular orientation of the spinal rod holder relative to the spine screw and to fix the spinal rod relative to the spinal rod holder.

2. The spinal rod reception assembly of claim 1, wherein fixing of the angular orientation of the spinal rod holder relative to the spine screw and of the spinal rod relative to the spinal rod holder is achieved simultaneously upon reception of the end cap in the spinal rod holder.

3. The spinal rod reception assembly of claim 1, wherein:
the axial bore of the spinal rod holder has internal threading; and
the end cap has external threading;
the internally threaded axial bore threadedly receiving the externally threaded end cap whereby through continued threading of the externally threaded end cap into the internally threaded axial bore, the externally threaded end cap provides the application of pressure on the first end of the insert after placement of the spinal rod in the spinal rod holder to fix the angular orientation of the spinal rod holder relative to the spine screw and to fix the spinal rod relative to the spinal rod holder.

4. The spinal rod reception assembly of claim 3, wherein the externally threaded end cap includes a detachable portion configured to drive the threading of the end cap into the spinal rod holder.

5. The spinal rod reception assembly of claim 4, wherein the detachable portion defines a driver.

6. The spinal rod reception assembly of claim 1, wherein:
the second end of the spinal rod holder is internally angled radially inward; and
the second end of the insert is angled for reception on the head portion of the spinal screw.

7. The spinal rod reception assembly of claim 6, wherein the insert forms a pocket for the reception of the spinal rod therein.

8. The spinal rod reception assembly of claim 7, wherein the first end of the insert is angled radially inward.

9. A spinal rod reception assembly comprising:
a spinal bone screw;
a spinal rod holder configured to hold a spinal rod and having an axial bore therein defining a first opening and a second opening, the second opening configured to hold a head portion of the spinal bone screw and to allow polyaxial pivoting of the spinal bone screw, the first opening configured to receive the spinal rod;
an insert received in the axial bore of the spinal rod holder and configured to receive the spinal rod, the insert defining a first end and a second end, the second end configured to abut the head portion of the spinal bone screw and to fix an angular orientation of the spinal bone screw relative to the spinal rod holder upon application of axial pressure on the first end of the insert, and the first end configured to fix a position of the spinal rod relative to the spinal rod holder upon application of the axial pressure on the first end of the insert; and
an end cap receivable in the axial bore of the spinal rod holder and configured to apply axial pressure on the first end of the insert after placement of the spinal rod in the spinal rod holder to fix the angular orientation of the spinal rod holder relative to the spine bone screw and to fix the spinal rod relative to the spinal rod holder.

10. The spinal rod reception assembly of claim 9, wherein fixing of the angular orientation of the spinal rod holder relative to the spine bone screw and of the spinal rod relative to the spinal rod holder is achieved simultaneously upon reception of the end cap in the spinal rod holder.

11. The spinal rod reception assembly of claim 9, wherein:
the axial bore of the spinal rod holder has internal threading; and
the end cap has external threading;
the internally threaded axial bore threadedly receiving the externally threaded end cap whereby through continued threading of the externally threaded end cap into the internally threaded axial bore, the externally threaded end cap provides the application of pressure on the first end of the insert after placement of the spinal rod in the spinal rod holder to fix the angular orientation of the spinal rod holder relative to the spine bone screw and to fix the spinal rod relative to the spinal rod holder.

12. The spinal rod reception assembly of claim 11, wherein the externally threaded end cap includes a detachable portion configured to drive the threading of the end cap into the spinal rod holder.

13. The spinal rod reception assembly of claim 12, wherein the detachable portion defines a driver.

14. The spinal rod reception assembly of claim 9, wherein:
the second end of the spinal rod holder is internally angled radially inward; and
the second end of the insert is angled for reception on the head portion of the spinal bone screw.

15. The spinal rod reception assembly of claim 14, wherein the insert forms a pocket for the reception of the spinal rod therein.

16. The spinal rod reception assembly of claim 15, wherein the first end of the insert is angled radially inward.

17. A spinal rod reception assembly for receiving and retaining a spinal rod therein, the spinal rod reception assembly comprising:
a vertebral bone screw;
a spinal rod holder configured to receive and hold a spinal rod, the spinal rod holder having a threaded axial bore therein defining a first opening and a second opening, the second opening configured to hold a head portion of the vertebral bone screw and to allow polyaxial pivoting of the vertebral bone screw, the first opening configured to receive the spinal rod;
a collet received in the threaded axial bore of the spinal rod holder and configured to also receive the spinal rod, the collet defining a first end and a second end, the second end configured to abut the head portion of the vertebral bone screw and to fix an angular orientation of the vertebral bone screw relative to the spinal rod holder upon application of axial pressure on the first end of the collet, and the first end configured to fix a position of the spinal rod relative to the spinal rod holder upon application of the axial pressure on the first end of the collet; and
a threaded lock-up cap threadedly receivable in the threaded axial bore of the spinal rod holder and configured to apply axial pressure on the first end of the collet after placement of the spinal rod in the spinal rod holder to fix the angular orientation of the spinal rod holder relative to the vertebral bone screw and to fix the spinal rod relative to the spinal rod holder.

18. The spinal rod reception assembly of claim 17, wherein fixing of the angular orientation of the spinal rod holder relative to the vertebral bone screw and of the spinal rod relative to the spinal rod holder is achieved simultaneously upon threaded reception of the threaded end cap in the spinal rod holder.

19. The spinal rod reception assembly of claim 18, wherein the externally threaded end cap includes a detachable driver configured to thread the threaded end cap into the spinal rod holder.

20. The spinal rod reception assembly of claim 17, wherein:
the second end of the spinal rod holder is internally angled radially inward;
the second end of the collet is angled for reception on the head portion of the vertebral bone screw; and
the collet forms a pocket for the reception of the spinal rod therein.
